(19) United States
(12) Patent Application Publication
(10) Pub. No.: US 2026/0363751 A1
(43) Pub. Date: Nov. 19, 2026

(54) ASYMMETRICAL SHAFT STRUT IDENTIFIER

(71) Applicant: Boston Scientific Scimed, Inc., Maple Grove, MN (US)

(72) Inventors: James HOLMBERG, Champlin, MN (US); Benjamin WILKE, Shoreview, MN (US)

(21) Appl. No.: 19/676,492

(22) Filed: May 13, 2026

Related U.S. Application Data
(60) Provisional application No. 63/805,187, filed on May 13, 2025.

(51) Int. Cl. A61B 18/00; A61B 34/20; A61B 90/00

(52) U.S. Cl. CPC A61B 18/00; A61B 34/20; A61B 90/39; A61B 2018/0016; A61B 2018/00267; A61B 2034/2051; A61B 2090/3966

(57) ABSTRACT

An electrosurgical medical device includes a tubular outer shaft having a proximal end and an opposite distal end. A lumen extends between the proximal end and the distal end. An electrode assembly extends distally from the distal end of the outer shaft. The electrode assembly has a distally located central hub portion and a plurality of splines. Each of the plurality of splines includes a distal end portion extending from the central hub portion, and a proximal end portion attached to the outer shaft. An asymmetrical marker is located on the outer shaft. The asymmetrical marker includes a transverse member, a distal flag extending distally from the transverse member, and a proximal flag extending proximally from the transverse member. The distal flag is aligned with one of the plurality of splines and the proximal flag is aligned with a second of the plurality of splines.

### CROSS REFERENCE TO RELATED APPLICATIONS

The present application claims priority to U.S. Provisional Patent Application No. 63/805,187, filed May 13, 2025, the entire disclosure of which is incorporated herein in its entirety.

### TECHNICAL FIELD

The present disclosure relates to medical systems and methods for performing an electrosurgical procedure in a patient. More specifically, the present disclosure relates to medical systems and methods having an asymmetrical marker for aiding alignment and navigation during an electrosurgical procedure.

### BACKGROUND

Electrosurgical procedures are used to treat many different conditions in patients. Ablation is one procedure that can be used to treat cardiac arrhythmias, benign tumors, cancerous tumors, and to control bleeding during surgery. Ablation is accomplished through various techniques including, for example, electroporation, thermal ablation (e.g., radio-frequency) and cryoablation. During an ablation procedure, a device (e.g., a catheter or probe) is inserted into the patient and energy is transmitted through electrodes located on the device to the surrounding tissue. Such devices may also include mapping electrodes for detecting electrical activity of the adjacent tissue.

Some mapping and ablation devices include expanding assemblies, or cages, having expandable members (e.g., splines) that carry one or more electrosurgical electrode for delivering energy to a patent. The devices may carry one or more additional electrode useable for sensing a parameter within the body, or for aiding in navigation during the procedure.

### SUMMARY

Example 1 is a medical device including an outer shaft having a proximal end and an opposite distal end. An electrode assembly extends distally from the distal end of the outer shaft. The electrode assembly has a distally located central hub portion and a plurality of splines. Each of the plurality of splines includes a distal end portion extending from the central hub portion, and a proximal end portion attached to the outer shaft. At least one electrode is located on each of the plurality of splines. An asymmetrical marker is located on the outer shaft. The asymmetrical marker has a transverse member, a distal flag extending distally from the transverse member, and a proximal flag extending proximally from the transverse member. The distal flag is aligned with one of the plurality of splines and the proximal flag is aligned with a second of the plurality of splines.

Example 2 is the medical device of Example 1, wherein the asymmetrical marker is formed of a radiopaque material viewable under imaging.

Example 3 is the medical device of any of Examples 1 or 2, wherein the transverse member is curved to conform to the outer shaft.

Example 4 is the medical device of any of Examples 1 to 3, wherein the transverse member is a ring that circumscribes the outer shaft.

Example 5 is the medical device of any of Examples 1 to 4, wherein the one of the plurality of splines is adjacent the second of the plurality of splines.

Example 6 is the medical device of Example 4, wherein the second of the plurality of splines is located clockwise from the one of the plurality of splines.

Example 7 is the medical device of Example 4, wherein the second of the plurality of splines is located counterclockwise from the one of the plurality of splines.

Example 8 is the medical device of any of Examples 1 to 4, wherein at least one spline is between the one of the plurality of splines and the second of the plurality of splines.

Example 9 is the medical device of any of Examples 1 to 8, wherein the outer shaft includes a sensor, and the distal flag is offset from the sensor.

Example 10 is the medical device of any of Examples 1 to 9, wherein the electrode assembly includes an expanded configuration, and the distal flag terminates proximally of a bend of the one of the plurality of splines in the expanded configuration.

Example 11 is the medical device of any of Examples 1 to 10, wherein the asymmetrical marker is at least partially embedded in the outer shaft.

Example 12 is the medical device of any of Examples 1 to 11, wherein the distal flag or the proximal flag includes at least one protrusion.

Example 13 is the medical device of any of Examples 1 to 12, wherein the distal flag is asymmetric along its length from the transverse member to a distal end.

Example 14 is the medical device of any of Examples 1 to 13, wherein the proximal flag is asymmetric along its length from the transverse member to a proximal end.

Example 15 is the medical device of any of Examples 1 to 11, wherein the proximal flag is symmetric along its length from the transverse member to a proximal end, and the distal flag is symmetric along its length from the transverse member to a distal end.

Example 16 is a medical device. The device includes a tubular outer shaft having a proximal end and an opposite distal end. A lumen extends between the proximal end and the distal end. An electrode assembly extends distally from the distal end of the outer shaft. The electrode assembly has a distally located central hub portion and a plurality of splines. Each of the plurality of splines includes a distal end portion extending from the central hub portion, and a proximal end portion attached to the outer shaft. At least one electrode is located on each of the plurality of splines. An asymmetrical marker is located on the outer shaft. The asymmetrical marker includes a transverse member, a distal flag extending distally from the transverse member, and a proximal flag extending proximally from the transverse member. The distal flag is aligned with one of the plurality of splines and the proximal flag is aligned with a second of the plurality of splines.

Example 17 is the medical device of Example 16, wherein the asymmetrical marker is formed of a radiopaque material viewable under imaging.

Example 18 is the medical device of Example 16, wherein the transverse member is curved to conform to the outer shaft.

Example 19 is the medical device of Example 16, wherein the transverse member is a ring that circumscribes the outer shaft.

Example 20 is the medical device of Example 16, wherein the one of the plurality of splines is adjacent the second of the plurality of splines.

Example 21 is the medical device of Example 20, wherein the second of the plurality of splines is located clockwise from the one of the plurality of splines.

Example 22 is the medical device of Example 20, wherein the second of the plurality of splines is located counterclockwise from the one of the plurality of splines.

Example 23 is the medical device of Example 16, wherein at least one spline is between the one of the plurality of splines and the second of the plurality of splines.

Example 24 is the medical device of Example 16, wherein the outer shaft includes a sensor, and the distal flag is offset from the sensor.

Example 25 is the medical device of Example 16, wherein the electrode assembly includes an expanded configuration, and the distal flag terminates proximally of a bend of the one of the plurality of splines in the expanded configuration.

Example 26 is the medical device of Example 16, wherein the asymmetrical marker is at least partially embedded in the outer shaft.

Example 27 is the medical device of Example 16, wherein the distal flag or the proximal flag includes at least one protrusion.

Example 28 is the medical device of Example 16, wherein the distal flag is asymmetric along its length from the transverse member to a distal end.

Example 29 is the medical device of Example 16, wherein the proximal flag is asymmetric along its length from the transverse member to a proximal end.

Example 30 is the medical device of Example 16, wherein the proximal flag is symmetric along its length from the transverse member to a proximal end, and the distal flag is symmetric along its length from the transverse member to a distal end.

Example 31 is an electrosurgical medical device. The device includes an outer shaft having a proximal end and an opposite distal end. An electrode assembly extends distally from the distal end of the outer shaft. The electrode assembly has a distally located central hub portion and a plurality of splines. Each of the plurality of splines includes a distal end portion extending from the central hub portion, and a proximal end portion attached to the outer shaft. At least one electrode is located on each of the plurality of splines. An asymmetrical marker is at least partially embedded in the outer shaft. The asymmetrical marker includes a transverse member, a distal flag extending distally from the transverse member, and a proximal flag extending proximally from the transverse member. The distal flag is aligned with one of the plurality of splines and the proximal flag is aligned with a second of the plurality of splines.

Example 32 is the medical device of Example 31, wherein the distal flag is asymmetric along its length from the transverse member to a distal end, or the proximal flag is asymmetric along its length from the transverse member to a proximal end.

Example 33 is the medical device of Example 31, wherein the proximal flag is symmetric along its length from the transverse member to a proximal end, and the distal flag is symmetric along its length from the transverse member to a distal end.

Example 34 is an electrosurgical medical device. The device includes a tubular outer shaft having a proximal end and an opposite distal end. A lumen extends between the proximal end and the distal end of the outer shaft. An electrode assembly extends distally from the distal end of the outer shaft. The electrode assembly has a plurality of splines. Each of the plurality of splines includes at least one electrode and a proximal end portion attached to the outer shaft. An asymmetrical marker is located on the outer shaft. The asymmetrical marker includes a transverse member including a curve configured to conform to the outer shaft. A distal flag extends distally from the transverse member, and a proximal flag extends proximally from the transverse member. The distal flag is aligned with one of the plurality of splines and the proximal flag is aligned with an adjacent spline.

Example 35 is the medical device of Example 34, wherein the asymmetrical marker is formed of a radiopaque material viewable under imaging.

In some situations, it may be difficult to properly orient the splines for delivery of energy to a desired tissue location. The present disclosure aims to provide an asymmetrical marker that allows for determination of orientation of splines. While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, using an electrophysiology system.

FIG. 2A is a side view of an electrosurgical catheter suitable for use in the electrophysiological system of FIG. 1, in which the catheter is in a collapsed configuration.

FIG. 2B is a perspective view of a distal portion of an electrosurgical catheter with an expandable assembly in a stored configuration, in accordance with embodiments of the disclosure.

FIGS. 2C and 2D illustrate perspective views of a distal portion of an electrosurgical catheter with an expandable assembly in various expanded configurations, in accordance with embodiments of the disclosure.

FIG. 3A illustrates a plan view of an asymmetrical marker for use with the electrosurgical catheter of FIG. 2A, in accordance with embodiments of the disclosure.

FIGS. 3B-3D illustrate end views of various embodiments of an asymmetrical marker of FIG. 3A, in accordance with embodiments of the disclosure.

FIG. 4A illustrates a plan view of an asymmetrical marker for use with the electrosurgical catheter of FIG. 2A, in accordance with embodiments of the disclosure.

FIGS. 4B-4D illustrate end views of various embodiments of an asymmetrical marker of FIG. 4A, in accordance with embodiments of the disclosure.

FIG. 5 illustrates imaging of a sequence of rotating the catheter of FIG. 2A including an asymmetrical marker, in accordance with embodiments of the disclosure.

FIGS. 6A-6C illustrates plan views of embodiments of an asymmetrical marker having asymmetric flags for use with the catheter of FIG. 2A, in accordance with embodiments of the disclosure.

FIGS. 7A-7E illustrates plan views of embodiments of an asymmetrical marker having flags including at least one protrusion for use with the catheter of FIG. 2A, in accordance with embodiments of the disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

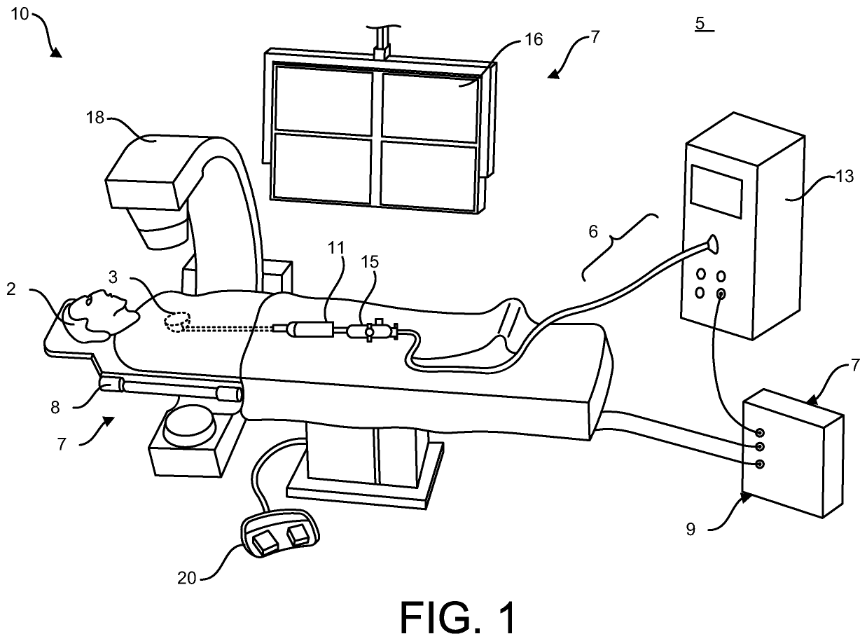

FIG. 1 illustrates an example clinical setting 10 for treating a patient 2, such as for treating a heart 3 of the patient 2, using an electrophysiology system 5, in accordance with the disclosure. The electrophysiology system 5 includes an electroporation device (e.g., an electroporation catheter system 6 and an electro-anatomical mapping (EAM) system 7. While the present disclosure focuses on an electroporation device, this is just one exemplary embodiment. The techniques disclosed herein are also applicable to any medical device including a distal element with expandable components (e.g., splines).

As shown in FIG. 1, the example electroporation catheter system 6 includes an electroporation catheter 15 (also known as an electrosurgical catheter 15), an introducer sheath 11, and an electroporation console 13. Additionally, the electroporation catheter system 6 includes various connecting elements, such as cables, that operably connect the components of the electroporation catheter system 6 to one another and to the components of the EAM system 7. In general, the EAM mapping system 7 includes a localization field generator 8, a mapping and navigation controller 9, and a display 16. Also, the clinical setting 10 can include additional equipment such as imaging equipment 18 (represented by the C-arm) and various controller elements, such as a foot controller 20, configured to allow an operator to control various aspects of the electrophysiology system 5. The clinical setting 10 may have other components and arrangements of components that are not shown in FIG. 1. Other arrangements of connecting elements, including wireless connecting elements, are contemplated.

The electroporation catheter system 6 is configured to deliver electric field energy to targeted tissue in the patient's heart 3 to create cell death in tissue, for example, rendering the tissue incapable of conducting electrical signals. Also, the electroporation catheter system 6 is configured to generate, based on models of electric fields, graphical representations of the electric fields that can be produced using the electroporation catheter 15 and to overlay, on the display 16, the graphical representations of the electric fields or expected or predicted lesions on an anatomical map of the patient's heart to aid a user in planning ablation by irreversible electroporation using the electroporation catheter 15 prior to delivering energy. In embodiments, the electroporation catheter system 6 is configured to generate the graphical representations of the electric fields based on characteristics of the electroporation catheter 15 and the position of the electroporation catheter 15 in the patient 2, such as in the heart 3 of the patient 2. The electroporation catheter system 6 is configured to generate the graphical representations of the electric fields based on characteristics of the electroporation catheter 15 and the position of the electroporation catheter 15 in the patient 2, such as in the heart 3 of the patient 2, and the characteristics of the tissue surrounding the catheter 15, such as measured impedances of the tissue.

The introducer sheath 11 is operable to provide a delivery conduit through which the electroporation catheter 15 can be deployed to the specific target sites within the patient's heart 3. Access to the patient's heart can be obtained through a vessel, such as a peripheral artery or vein. Once access to the vessel is obtained, the electroporation catheter 15 can be navigated to within the patient's heart, such as within a chamber of the heart.

The EAM system 7 is operable to track the location of the various components of the electroporation catheter system 6, and to generate high-fidelity three-dimensional anatomical and electro-anatomical maps of the heart, including portions of the heart such as cardiac chambers of interest or other structures of interest such as the sinoatrial node or atrioventricular node. In one illustrative example, the EAM system 7 can include the OPAL HDx™ Mapping System marketed by Boston Scientific Corporation. Also, the mapping and navigation controller 9 of the EAM system 7 includes one or more controllers, such as microprocessors or computers, that execute code out of memory to control or perform functional aspects of the EAM system 7, in which the memory, can be part of the one or more controllers, microprocessors, computers, or part of a memory device accessible through a computer network.

The EAM system 7 generates a localization field, via the field generator 8, to define a localization volume about the heart 3, and a location sensor or sensing element on a tracked device, such as sensors on the electroporation catheter 15, generate an output that can be processed by the mapping and navigation controller 9 to track the location of the sensor, and consequently, the corresponding device, within the localization volume. In the illustrated example, the device tracking is accomplished using magnetic tracking techniques, in which the field generator 8 is a magnetic field generator that generates a magnetic field defining the localization volume, and location sensors on the tracked devices are magnetic field sensors.

In other examples, impedance tracking methodologies may be employed to track the locations of the various devices. In such examples, the localization field is an electric field generated, for example, by an external field generator arrangement, such as surface electrodes, by intra-body or intra-cardiac devices, such as an intracardiac catheter, or both. In these examples, the location sensing elements can constitute electrodes on the tracked devices that generate outputs received and processed by the mapping and navigation controller 9 to track the location of the various location sensing electrodes within the localization volume.

The EAM system 7 can be equipped for both magnetic and impedance tracking capabilities. In such examples, impedance tracking accuracy can, in some instances be enhanced by first creating a map of the electric field induced by the electric field generator within the cardiac chamber of interest using a probe equipped with a magnetic location sensor, as is possible using the OPAL HDx™ Mapping System.

Regardless of the tracking methodology employed, the EAM system 7 utilizes the location information for the various tracked devices, along with cardiac electrical activity acquired by, for example, the electroporation catheter 15 or another catheter or probe equipped with sensing electrodes, to generate, and display via the display 16, detailed three-dimensional geometric anatomical maps or representations of the heart tissue and voids such as cardiac chambers as well as electro-anatomical maps in which cardiac electrical activity of interest is superimposed on the geometric anatomical maps. Furthermore, the EAM system 7 can generate a graphical representation of the various tracked devices within the geometric anatomical map or the electro-anatomical map.

During a signal-acquisition stage of a cardiac mapping procedure, the catheter 15 is displaced to multiple locations within the heart chamber into which the catheter 15 is inserted. At each location to which the catheter 15 is moved, the electrodes and sensors acquire physiological signals resulting from the electrical activity in the heart along with positional, or spatial, information of the catheter 15. The spatial information is used in building a three-dimensional grid of the anatomy during mapping. To perform a mapping procedure and reconstruct physiological information on the endocardium surface, the EAM system 7 may align a coordinate system of the catheter 15 with the endocardium surface's coordinate system, or vice versa. Alternatively, or additionally, the grid may be used to capture EGMs and select mapping values based on statistical distributions associated with nodes of the grid. The EAM system 7 also can perform post-processing operations on the physiological information to extract and display useful features of the information to the operator of the system 5.

In generating an example electroanatomical map, a data stream including multiple signals, such as signals received from the mapping electrodes of the catheter 15, is input into the EAM system 7. During the automated electroanatomical mapping process, the data stream provides a collection of physiological and location signals that serve as an input to the mapping process. The signals may be collected directly by the mapping system, obtained from another system using an analog or digital interface, or both. The data stream can include signals such as unipolar and/or bipolar intracardiac EGMs, surface electrocardiograms (ECGs), electrode location information originating from one or more of a variety of methodologies, tissue proximity information, catheter force information, catheter to tissue contact information, catheter temperature, acoustic information, catheter electrical coupling information, catheter deployment shape information, electrode properties, respiration phase, blood pressure, and other physiological information. For the generation of specific types of maps, one or more signals may be used as one or more references to trigger and align the data stream relative to a cycle or clock, which can be used to create beat datasets. Beat metrics can be determined from the beat datasets. A beat acceptance process can be applied to determine which beat datasets will make up a map dataset. The map dataset may be stored in association with a three-dimensional grid that is dynamically generated during data acquisition.

Surface geometry data of the cardiac surface is generated, such as generated concurrently, during the data acquisition process using acceptance metrics employing a surface geometry construction process. This process constructs surface geometry using data such as electrode locations and catheter shape contained in the data stream. Additionally, or alternatively, previously collected surface geometry of the cardiac surface can be used as an input to surface geometry data. Previously collected geometry may have been collected using a different map dataset or using a different modality such as computerized tomography (CT), magnetic resonance imaging (MRI), ultrasound, or rotational angiography and registered to the catheter locating system. A surface map generation process is employed to generate surface map data from the map dataset and surface geometry data.

The depiction of the electrophysiology system 5 shown in FIG. 1 is intended for illustration or a general overview of the various components of the system 5 and is not intended to imply that the disclosure is limited to any set of components or arrangement of the components. For example, additional hardware components, such as breakout boxes or workstations, can be included in the electrophysiology system 5.

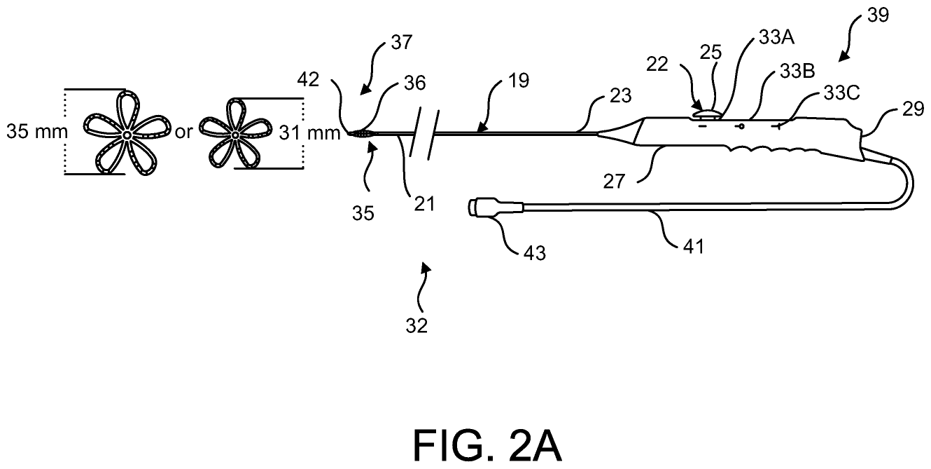

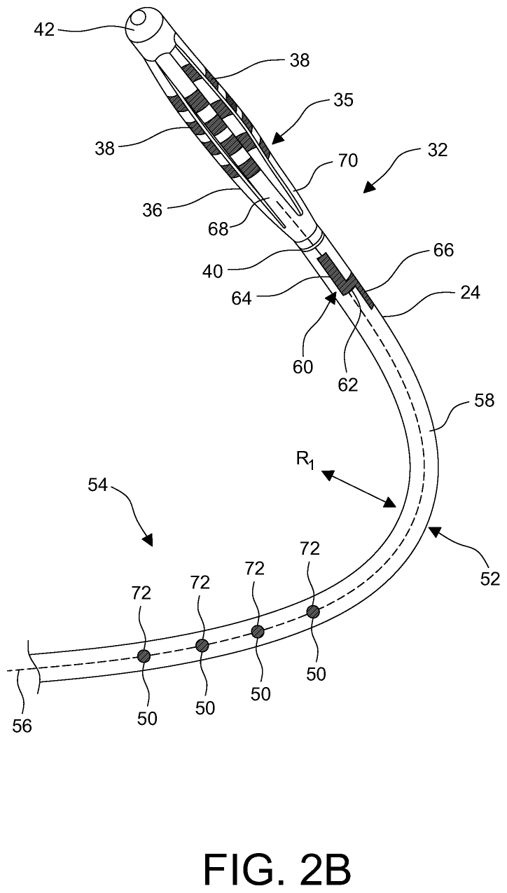

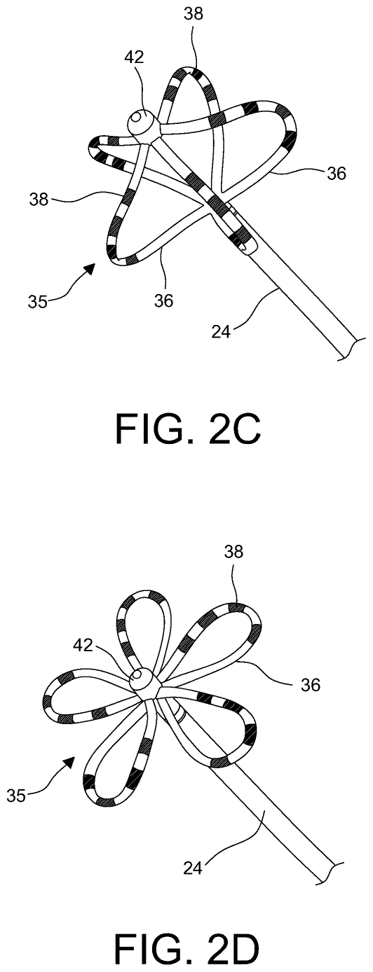

FIG. 2A is a side view of an embodiment of a mapping and ablation catheter 32, which corresponds with catheter 15 of FIG. 1. The catheter 32 can assume a plurality of configurations including a collapsed configuration and an expanded configuration. FIG. 2A illustrates the catheter 32 in the collapsed configuration. As shown in FIG. 2B, the catheter 32 includes an electrode array 35 comprising a set of splines 36 at a distal end 37 of the catheter 32 and a handle 27 at a proximal end 39 of the catheter 32. Each spline of the set of splines 36 includes one or more electrodes 38 formed on a surface of the spline. The electrodes can be configured as ablation electrodes for performing an ablation, sensing electrodes for performing mapping or detection of physiological signals, either ablation or sensing electrodes, or a combination of ablation electrodes and sensing electrodes. Each spline of the set of splines 36 can be configured to include a flexible curvature so as to rotate, or twist and bend and form a petal or tear drop-shaped curve.

In the example of FIG. 2A, the catheter 32 includes a catheter shaft 19 defining a longitudinal axis. The shaft 19 includes a distal region 21 and a proximal region 23. A shaft lumen extends longitudinally through the shaft 19 from the distal region 21 to the proximal region 23 along the longitudinal axis. In some embodiments, a diameter of the catheter shaft 19 is between about 6 French and about 15 French. In certain embodiments, the catheter shaft 19 has a diameter of about 12 French. In some embodiments, the catheter shaft 19 has a length of between about 85 cm and about 135 cm. In certain embodiments, the catheter shaft has a diameter of about 115 cm.

In some examples, as shown in FIG. 2B, a distal cap 42 includes a guidewire lumen, and a guidewire extends along the longitudinal axis in the shaft lumen and through the guidewire lumen in the distal cap 42. The distal cap 42 includes an atraumatic shape in the illustration and can include an electrical component in some embodiments. The set of splines 36 extend from the distal region 21 of the shaft 19. In exemplary embodiments, the set of splines 36 includes a proximal end affixed to the distal region 21 of the shaft 19. The splines include distal ends tethered or fixed to the distal cap 42 such that the set of splines 36 and distal cap 42 can translate relative to the catheter shaft 19 so as to expand and contract together.

In various embodiments, the distal cap 42 translates longitudinally along the guidewire extending through the lumen of the shaft. The set of splines 36 are configured for translation along the longitudinal axis to transition between the collapsed configuration and the expanded configuration. In some examples, the set of splines are configurable in an intermediate configuration for use such as ablation and mapping. The intermediate configuration is between the collapsed configuration and the expanded configuration, and the set of splines can be arranged in, for example, a basket or spherical configuration.

As shown in FIG. 2A, the proximal region 23 of the catheter shaft 19 in some embodiments is coupled to a handle 27 at the proximal end 39 of the catheter 32. The handle 27 includes a guidewire lumen 29 therethrough. The handle 27 is operably coupled to the set of splines 36 and the distal cap 42. For example, the handle 27 includes an actuator 22 disposed in the handle 27. The actuator 22 can include a knob 25, such as a thumb-activated slider, configured to be manipulated translationally or rotationally by a clinician. In the illustrated embodiment, the actuator 22 is configured for translation along the longitudinal axis by moving or sliding the knob 25 between a plurality of positions 33A, 33B, 33C to transition the electrode array 35 between a set of configurations including a collapsed configuration at a collapsed configuration position 33A, the intermediate or basket configuration at an intermediate configuration position 33B, and the expanded configuration at an expanded configuration position 33C.

In another embodiment, the actuator 22 can be configured to rotated about the longitudinal axis to transition the catheter 32 between a lock state and an unlock state. The lock state can fix a translational position of the set of splines 36 and the distal cap 42 relative to the catheter shaft 19 and the unlock state can permit translation of the distal cap 42 and set of splines 36 relative to the catheter shaft 19. In some embodiments, the handle 27 includes a flush port (not shown) for saline irrigation. For example, a saline flow may be used to maintain a predetermined level of flow to prevent thrombus formation. The handle 27 can include a cable 41 having connector 43. In some embodiments, the cable 41 is relatively short (e.g., up to about one meter) to increase maneuverability and flexibility of the catheter 32. The connector 43 is configured to couple to an extension cable to connect the catheter 32 to a console, such as console 13, or other components. In some embodiments, connector 43 is electrically connected to electrical components such as the electrodes 38, impedance tracking sensors, a magnetic sensor, temperature sensor, a gyroscopic sensor, an accelerometer via leads disposed in the handle 27, shaft 19, or on the electrode array 35. In some embodiments, the cable 41 and connector 43 can be configured for optical or other signal types.

In some embodiments, the catheter shaft 19 includes an outer member 24 connected to a distal end 26 of the handle 27, and an inner member (not shown) longitudinally moveable within the outer member 24. Movement of the inner member relative to the outer member 24 moves the cap 42 and causes the electrode array 35 to move between a stored and an expanded configuration.

The actuator 22 is configured to be moved by a user's thumb. As the actuator 22 is moved longitudinally with respect to the housing 12, the inner member moves longitudinally within the outer member 24 to adjust the arrangement of the electrode array 35.

FIG. 2B illustrates an exemplary medical device in the form of an electrosurgical mapping and ablation catheter 32 for use in a medical procedure. The catheter 32 includes an electrode array 35, also referred to as an expandable assembly. The electrode array 35 includes a plurality of splines 36 each having at least one electrode 38. The plurality of splines 36 form a basket structure that positions the at least one electrode 38 against a tissue surface within a patent. In the embodiment illustrated in FIG. 2A-2D, the electrode array 35 includes five splines 36, and each of the five splines 36 includes four electrodes 38. Other embodiments include more or less splines 36 and more or less electrodes 38 to facilitate use in various locations of a patient and to achieve a desired purpose. The at least one electrode 38 can take the form of ablation electrodes, mapping electrodes, pacing electrodes, sensing electrodes, or cauterizing electrodes depending on a procedure to be performed. In some embodiments, all of the electrodes 38 are configured for simultaneous use. In some embodiments, each of the electrodes 38 are configured for individual use and can be energized independently of each other.

The outer member 24 of the catheter 32 includes a distal end 40 that is attached to a proximal end of the plurality of splines 36. A distal end of the plurality of splines 36 is attached to the distal cap 42. In some embodiments, the cap 42 is a separate component. In other embodiments, the cap 42 is formed by molding together the plurality of splines 36. The cap 42 is attached to a distal end of the inner member such that retraction of the inner member causes the plurality of splines 36 to move from a stored configuration as illustrated in FIGS. 2A and 2B to an expanded configuration as illustrated in FIGS. 2C and 2D.

In FIG. 2B, the stored configuration of the electrode array 35 corresponds to the actuator 22 being in a most distal position on the handle 27. In this arrangement, the inner member is also in a most distal position such that the plurality of splines 36 are substantially parallel to a longitudinal axis of the catheter 32.

In FIG. 2C, the electrode array 35 of the catheter 32 is in an expanded configuration where the splines 36 form a bow shape. This arrangement corresponds to the actuator 22 being retracted proximally from the most distal position, which in turn retracts the inner member.

In FIG. 2D, the electrode array 35 of the catheter 32 is in an expanded configuration where the splines 36 form a petal configuration. This arrangement corresponds to the actuator 22 being retracted to a most proximal position on the handle 27, and the inner member it its most proximal position.

The actuator 22 is capable of being moved from a most proximal position to a most distal position, and vice versa, by a user pushing or pulling the actuator. The electrode array 35 of the catheter 32 moves between the stored configuration to the petal configuration as the actuator 22 is translated. A user is able to leave the actuator 22 in any location between the most distal position and the most proximal position in order to expand the splines 36 a desired amount between the stored configuration and the petal configuration.

The catheter 32 is configured to bend along a bending portion 52 that is located proximal of the electrode array 35. The bending portion 52 allows for the electrode array 35 to be directed towards a desired location within a patient, for example an interior of a heart. The bending portion 52 includes a curve having an inner radius R₁ and an outer curve 58. In some embodiments, the inner radius R₁ is fixed. In other embodiments, the inner radius R₁ is adjustable. In some embodiments, the bending portion 52 can include a pre-formed bend having a desired configuration. In other embodiments, the catheter 32 is steerable in response to tension being applied to one or more steering wires (now shown) which extend from near the distal end 40 to the handle 27. In response to tension, the catheter 32 is configured to bend at the bending portion 52 to adjust the shape of the catheter 32. In some embodiments, the catheter 32 is steerable in one direction using a single steering wire. In other embodiments, the catheter 32 is steerable in two opposing directions by two opposing steering wires. In other embodiments, the catheter 32 is steerable in any direction by four uniformly spaced steering wires.

The outer member 24 of the catheter 32 includes at least one electrode 50 located in an intermediate region 54 that is proximal of the bending portion 52. As illustrated in FIG. 2B, the at least one electrode 50 includes four electrodes located along a length of the outer member 24. The at least one electrode 50 is configured as a sensing electrode, navigating electrode, or sensor. The at least one electrode 50 is electrically connected to a control system, for example an electroanatomical mapping system 7. At least one conductor (not shown in FIG. 2B) is electrically connected with the at least one electrode 50 for transferring electrical signals between the at least one electrode 50 and the control system. The at least one electrode 50 is configured as a partial electrode, ring electrode, or an embedded electrode as described below.

In some embodiments, the at least one electrode 50 comprises a plurality of electrodes sharing a common longitudinal axis 56 along the outer member 24. In FIG. 2B, the common longitudinal axis 56 is a neutral axis. A neutral axis is one that resides in a plane that is not in a bending direction of the catheter 32. The neutral axis resides in a plane that is orthogonal to a plane passing through the inner radius R₁ and an outer curve 58. In some embodiments, a plurality of electrodes share a common longitudinal axis that is opposite of a bend direction of the elongated medical device. In FIG. 2B, this axis extends along an outer curve 58 of the bending portion 52.

An asymmetrical marker 60 formed of a radiopaque material viewable under imaging is located on the outer shaft 24 to aid alignment and navigation of the electrode array 35 during an electrosurgical procedure. The shape of the asymmetrical mater 60 allows for inferring positioning of the electrode array 35 while using 2D fluoroscopy or other imaging modalities. The asymmetrical marker 60 is positioned near the shaft distal end 40 proximal of the electrode array 35. The distal flag 64 terminates proximally of a bend in the plurality of splines 36 in the expanded configuration so as to not interfere with expanding of the electrode array 35.

The asymmetrical marker 60 includes a transverse member 62, a distal flag 64 extending distally from the transverse member 62, and a proximal flag 66 extending proximally from the transverse member 62. The distal flag 64 is aligned with one of the plurality of splines and the proximal flag 66 is aligned with a second of the plurality of splines.

In FIG. 2B, the distal flag 64 is aligned with first spline 68 and the proximal flag 66 is aligned with adjacent second spline 70. That is, the distal flag 64 has a longitudinal axis that is common with a longitudinal axis of the first spline 68 and the proximal flag 66 has a longitudinal axis that is common with a longitudinal axis of the second spline 70. In FIG. 2B, the second spline 70 is located counterclockwise from the first spline 68. However, in other embodiments, the asymmetrical marker is configured to have a proximal flag 66 aligning with a second spline that is located clockwise from the first spline 68. In FIG. 2B, the second spline 70 is adjacent the first spline 68. However, in other embodiments, at least one spline is between the first spline 70 and the second spline aligned with the proximal flag 66.

In FIG. 2A, the distal flag 64 is aligned with the first spline 68 along common longitudinal axis 56. As such, the distal flag 64 is also aligned with the at least one electrode 50, as is the first spline 68. In some embodiments, the distal flag is 64 is offset from the at least one electrode 50, as is the first spline 68.

In some embodiments, the asymmetrical marker 60 is affixed to the outer surface of the outer shaft 24 using adhesives, welding, or other bonding methods. In some embodiments, the asymmetrical marker 60 is at least partially embedded in the outer shaft 24 during a reflow procedure. For example, in some embodiments, the transverse member 62 is embedded in a polymer forming the outer shaft while the distal flag 64 and the proximal flag 66 remain above the surface of the polymer. In other embodiments, the entire asymmetrical marker 60 is embedded in the polymer such that the marker 60 is completely encased by polymer.

The asymmetrical marker 60 is formed of a suitable biocompatible material, such as stainless steel, tantalum, titanium, gold, platinum, palladium, various alloys, such as platinum iridium, and nickel titanium (e.g., Nitinol), as well as ceramics and metal oxides that can be compressed into specific shapes or configurations. Additionally, the marker 60 can be formed of a doped polymer configured to be viewable under imaging. The marker 60 can be formed by cutting out a flat sheet of material into a desired shape and bending the marker 60 to correspond to a curve of the outer sheath 24. Alternatively, the marker 60 can be formed by a molding, casting, or other technique.

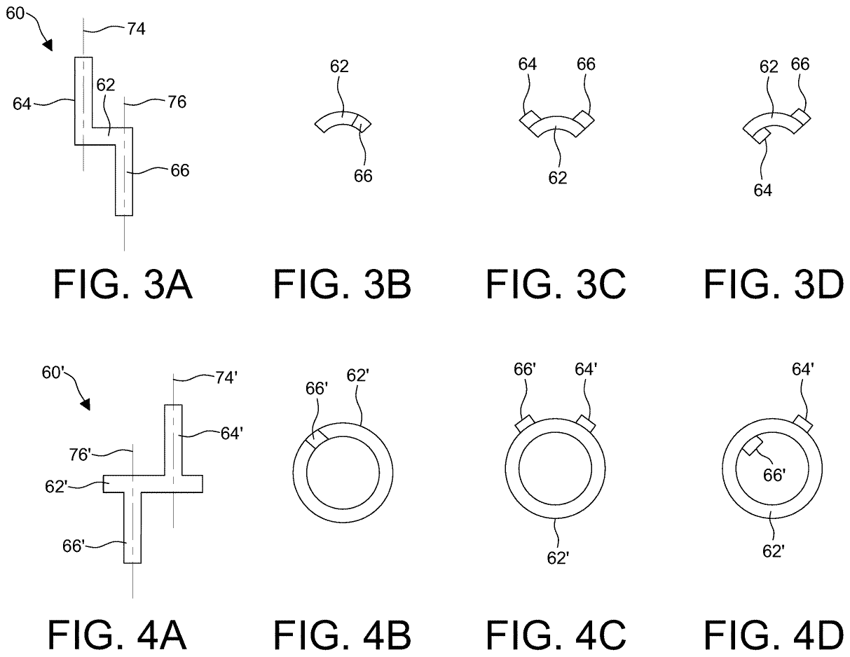

FIG. 3A illustrates a plan view of an asymmetrical marker 60 for use with the catheter of FIG. 2A, in accordance with embodiments of the disclosure. As illustrated in FIG. 3A, the asymmetrical marker 60 has a transverse member 62, a distal flag 64 extending distally from the transverse member 62, and a proximal flag 66 extending proximally from the transverse member 62. A longitudinal axis 74 of the distal flag 64 is parallel to a longitudinal axis 76 of the proximal flag 66. As discussed above, the longitudinal axis 74 of the distal flag 64 is aligned with one of the plurality of splines 36 and the longitudinal axis 76 of the proximal flag 66 is aligned with a second of the plurality of splines 36. The asymmetrical marker 60 includes a substantially S-shape configuration.

FIGS. 3B-3D illustrate end views of various embodiments of an asymmetrical marker 60 of FIG. 3A, in accordance with embodiments of the disclosure. As shown in FIGS. 3B-3D, the transverse member 62 is curved to conform to the outer shaft 24. The transverse member 62 only partially surrounds a portion of the outer shaft 24. In FIG. 3B, the distal flag 64, transverse member 62, and the proximal flag 66 all reside in a curved plane. This arrangement can be formed by molding, cutting the asymmetric marker 60 out of a flat sheet of material then bending to create the curve, or by cutting the asymmetric marker 60 out of a curved piece of material. In FIG. 3C, the distal flag 64 and the proximal flag 66 are on top of the transverse member 62. This arrangement can be formed by affixing a separate distal flag 64 and a separate proximal flag 66 to an upper surface of the transverse member 62. In FIG. 3D, the distal flag 64 is affixed to a lower surface of the transverse member 62 while the proximal flag 66 is affixed to an upper surface of the transverse member 62.

FIG. 4A illustrates a plan view of an asymmetrical marker 60′ for use with the catheter of FIG. 2A, in accordance with embodiments of the disclosure. As illustrated in FIG. 4A, the asymmetrical marker 60′ has a transverse member 62′, a distal flag 64′ extending distally from the transverse member 62′, and a proximal flag 66′ extending proximally from the transverse member 62′. A longitudinal axis 74′ of the distal flag 64′ is parallel to a longitudinal axis 76′ of the proximal flag 66′. As discussed above, the longitudinal axis 74′ of the distal flag 64′ is aligned with one of the plurality of splines 36 and the longitudinal axis 76′ of the proximal flag 66′ is aligned with a second of the plurality of splines 36.

FIGS. 4B-4D illustrate end views of various embodiments of an asymmetrical marker 60′ of FIG. 4A, in accordance with embodiments of the disclosure. As shown in FIGS. 4B-4D, the transverse member 62′ is a ring that completely circumscribes the outer shaft 24. In FIG. 4B, the distal flag 64', transverse member 62', and the proximal flag 66′ all reside in a curved plane. In FIG. 4C, the distal flag 64′ and the proximal flag 66′ are located on an outer surface of the transverse member 62'. This arrangement can be formed by affixing a separate distal flag 64′ and a separate proximal flag 66′ to an outer surface of the transverse member 62'. In FIG. 4D, the proximal flag 66′ is affixed to an inner surface of the transverse member 62′ while the distal flag 64′ is affixed to an outer surface of the transverse member 62'.

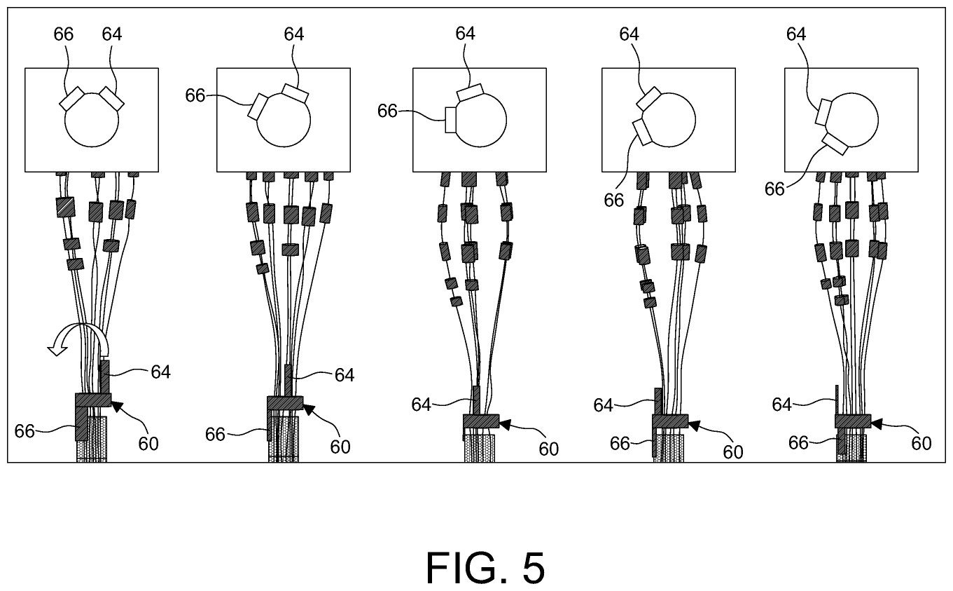

FIG. 5 illustrates imaging of a sequence of rotating the catheter 32 of FIG. 2A including an asymmetrical marker 60, in accordance with embodiments of the disclosure. As the catheter 32 is rotated, the asymmetrical marker 60 allows a user to determine a location and orientation of the electrode array 35.

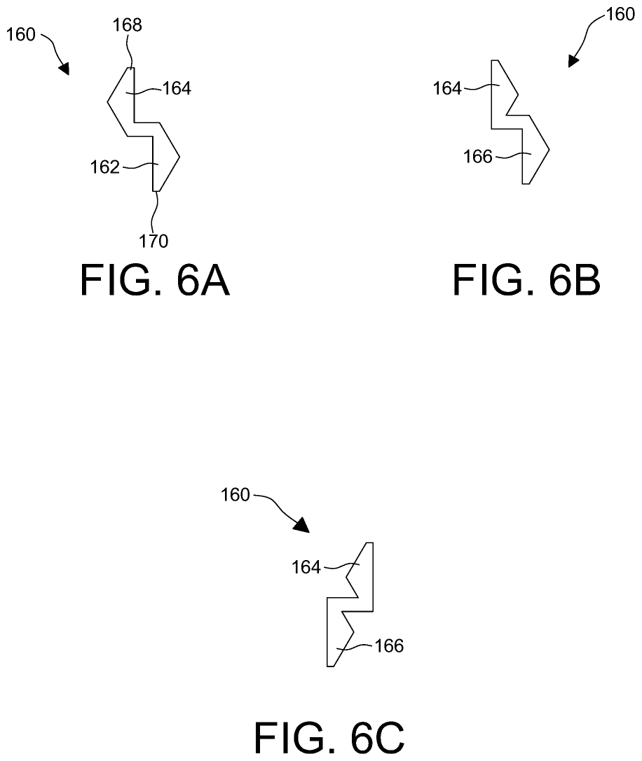

FIGS. 6A-6C illustrates plan views of embodiments of an asymmetrical marker 160 having asymmetric flags for use with the catheter of FIG. 2A, in accordance with embodiments of the disclosure. In FIGS. 6A-6C, the asymmetrical marker 160 includes a distal flag 164 that is asymmetric along its length from the transverse member 162 to a distal end 168. Also, the proximal flag 166 is asymmetric along its length from the transverse member 162 to a proximal end 170. In FIG. 6A, the distal flag 164 is configured as a triangle having an apex pointing in a first direction and the proximal flag is configured as a triangle having an apex pointing in a second direction that is opposite and away from the first direction. In FIG. 6B, the distal flag 164 is configured as a triangle having an apex pointing in the same direction that a triangle of the proximal flag is pointing. In FIG. 6C, the distal flag 164 is configured as a triangle having an apex pointing in a first direction and the proximal flag is configured as a triangle having an apex pointing in a second direction towards the first direction.

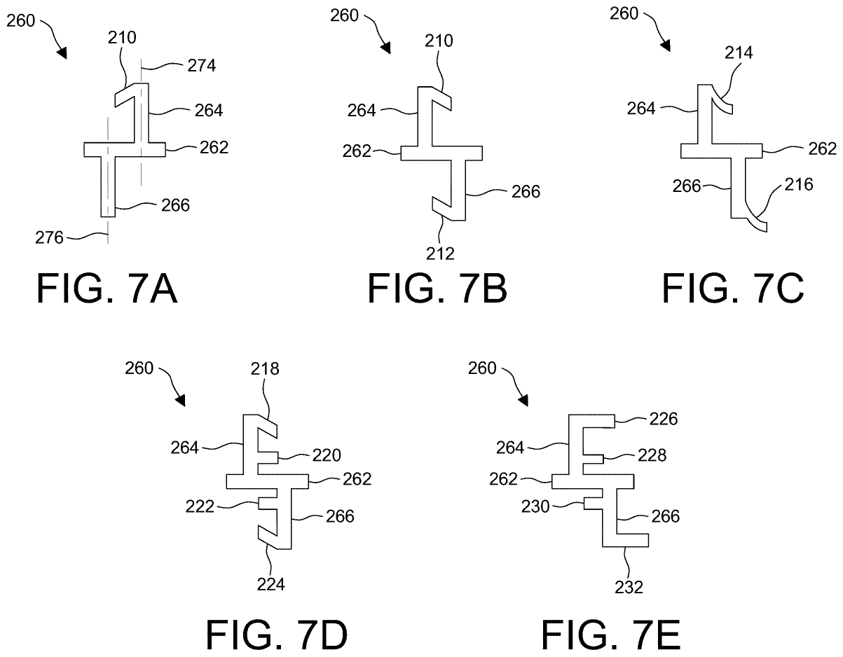

FIGS. 7A-7E illustrates plan views of embodiments of an asymmetrical marker 260 having flags including at least one protrusion for use with the catheter 32 of FIG. 2A, in accordance with embodiments of the disclosure. In FIGS. 7A-7E, the asymmetrical marker 260 includes a transverse member 262, a distal flag 264 extending distally from the transverse member 262, and a proximal flag 266 extending proximally from the transverse member 262. A longitudinal axis 274 of the distal flag 264 is parallel to a longitudinal axis 276 of the proximal flag 266. As discussed above, the longitudinal axis 274 of the distal flag 264 is aligned with one of the plurality of splines 36 and the longitudinal axis 276 of the proximal flag 266 is aligned with a second of the plurality of splines 36.

In FIG. 7A, the distal flag 264 includes a protrusion 210, and is therefore asymmetric along its length, while the proximal flag 266 is symmetric along its length. The protrusion 210 aids in determining orientation of the spines 36 of the electrode array 35 during a procedure. The protrusion 210 extends from an edge of the distal flag 265. In FIG. 7A, the protrusion 210 extends from an edge of the distal flag 265 at an angle. In FIG. 7B, the proximal flag 266 also includes a protrusion 212. The protrusion 212 of the proximal flag 266 is substantially identical to the protrusion 210 of the distal flag 264. The protrusion 210 of the distal flag 264 points in a first direction and the protrusion 212 of the proximal flag 266 points in a second direction.

In FIG. 7C, the distal flag 264 includes a curved protrusion 214 extending from an edge thereof and the proximal flag 266 also includes a curved protrusion 216 extending from an edge thereof. The curved protrusion 214 of the distal flag 264 points in the same direction as the curved protrusion 216 of the proximal flag 266.

In FIG. 7D, the distal flag 264 includes a plurality of protrusions 218, 220 pointing in a first direction. The plurality of protrusions 218, 220 include an angled protrusion 218 extending at an angle from an edge of the distal flag 264 as well as an orthogonal protrusion 220 that extends orthogonally from an edge of the distal flag 264. The proximal flag 266 also includes a plurality of protrusions 222, 224 pointing in a second direction. The plurality of protrusions 222, 224 include an angled protrusion 224 extending at an angle from an edge of the distal flag 264 as well as an orthogonal protrusion 222 that extends orthogonally from an edge of the distal flag 264.

In FIG. 7E, the distal flag 264 includes a plurality of protrusions 226, 228 pointing in a first direction. The plurality of protrusions 226, 228 include a longer orthogonal protrusion 226 and a shorter orthogonal protrusion 228 extending orthogonally from an edge of the distal flag 264. The shorter orthogonal protrusion 228 is thinner than the longer orthogonal protrusion 226. The proximal flag 266 also includes a plurality of protrusions 230, 232. The plurality of protrusions 230, 232 include a longer orthogonal protrusion 232 and a shorter orthogonal protrusion 230. The longer orthogonal protrusion 232 extends from an edge of the proximal flag 266 and points in the first direction. The shorter orthogonal protrusion 230 extends from an edge of the proximal flag 266 and points in a second direction opposite the first direction.

The example electroporation catheter 15 includes an elongated catheter shaft and distal end configured to be deployed proximate target tissue, such as within a chamber of the patient's heart. The distal end includes an electrode array to effect treatment. The catheter 15 is capable of being formed into a plurality of configurations. For example, if the distal end region of the catheter is within the patient's vasculature or is within a sheath as a catheter assembly, such as to travel to the patient to the chamber of the heart, the electrode array is in a collapsed configuration to fit within the sheath. Once the catheter has reached the destination in the chamber of the heart, for example, or the sheath is retracted from the distal region of the catheter 15 (or the shaft catheter is extended past the sheath), and the electrode array is arranged in an expanded configuration for use. In one embodiment, the electrode array can assume other configurations, such as an intermediate configuration between the collapsed and expanded configurations, such as an additional use configuration.

The electrode array includes an electrode assembly comprising a plurality of electrodes. For example, the electrode assembly includes a plurality of spaced-apart electrodes or multiple spaced-apart sets or groups of spaced-apart electrodes. In some examples, an electrode, such as a plurality of spaced-apart electrodes, can be deployed on the catheter shaft in addition to electrodes on the electrode array. In one example, the plurality of electrodes can be formed of a conductive, solid-surface, biocompatible material and are spaced-apart across electrical insulators. Each of the plurality of electrodes is electrically coupled to an associated elongated lead conductor that extend along the shaft to a catheter proximal end. In one example, each electrode of the spaced-apart electrodes corresponds with a separate, single lead conductor. In another example, a plurality of electrodes may be coupled to a single lead conductor. Other configurations are contemplated. The plurality of lead conductors can be electrically insulated from one another within an electrically insulating sheath along the catheter shaft, such as with an electrically insulating polymer sheath. The lead conductors can be electrically coupled to plug in the proximal region of the electroporation catheter 15, such as a plug configured to be mechanically and electrically coupled to the electroporation console 13 and the EAM system 7, for example, either directly or via intermediary electrical conductors such as cabling.

The electrode assemblies and associated electrodes are configured for, among other things, sensing cardiac electrical signals, ablation, localization of the electrode assembly within the patient anatomy such as via the EAM system 7, signal reference, and to determine proximity to target tissue within the anatomy. In some embodiments, the catheter 15 is configured for cardiac mapping, and the electrodes are sensing, or mapping, electrodes configured to be used to collect physiological (electrical) signals to be used to generate electroanatomical maps. An example of a physiological signal that the sensing electrode can acquire includes an intracardiac electrogram (ECG) signal. In some embodiments, the catheter 15 can be a mapping and ablation catheter, and the electrodes can include ablation electrodes, or an ablation electrode assembly, that are configured to deliver ablation electric field energy and sensing electrodes, or a sensing electrode assembly, for mapping purposes. The ablation electrodes in embodiments of an electroporation catheter are configured to receive pulsed electrical signals or waveforms from the console 13 and create pulsed electric fields sufficient to ablate target tissue via irreversible electroporation. The sensing electrodes in the electrode assembly can be electrically coupled to a one or more lead conductors that extends the length of the shaft that are configured to carry an electrical signal received at the sensing electrode. In some examples, an electrode in the electrode assembly can be configured to only perform an ablation or the electrode in the electrode assembly can be configured to only perform mapping. In some examples, an electrode can operate as an ablation electrode in an ablation mode of the electrophysiology system 5 and as a sensing electrode in a sensing or mapping mode of the system 5. Some examples of mapping and ablation catheters are smaller in profile or in the volume of the electrode assembly than catheters that just perform mapping, and clinicians can map a given location within the heart with fewer passes across the chamber with mapping catheters than with mapping and ablation catheters.

In one example, the electroporation console 13 is configured to provide an electrical signal, such as a plurality of concurrent or space-apart-time electrical signals, to the electrically connected electroporation catheter 15 along lead conductors to the spaced-apart electrodes. The spaced-apart electrodes are configured to generate a selected electrical field proximate the target tissue, based on the electrical signals from the electroporation console 13, to effect electroporation.

A selected electrical field can be generated with the electrodes configured as ablation electrodes to effect electroporation. A first ablation electrode, or first group of ablation electrodes, can be selected to be an anode and a different, second ablation electrode, or second group of ablation electrodes, can be selected to be a cathode, such that electrical fields can be generated between the anode and cathode based on signals, such as pulses, provided to the ablation electrodes from the electroporation console 13. The console 13 provides electric pulses of different lengths and magnitudes to the ablation electrodes on the catheter 15. The electric pulses can be provided in a continuous stream of pulses or in multiple, separate trains of pulses. Pulse parameters of interest include the number of pulses, the duty cycle of the pulses, the spacing of pulse trains, the voltage or magnitude of the pulses including the peak voltages, and the duration of the voltages. For example, the console 13 can select two or more ablation electrodes of the electrode assembly and provides pulses to the selected electrodes to generate electric fields between the selected electrodes to provide pulsed field ablation (PFA). For example, PFA can be performed with monophasic waveforms and biphasic waveforms. Without being bound to a particular theory, electric field strengths in the range of generally 200-25 volts per centimeter (V/cm) with microsecond-scale pulse duration have been demonstrated to provide reversible electroporation in cardiac tissue. Electric field strengths at approximately 400 V/cm have been demonstrated to provide irreversible electroporation in cardiac tissue of interest, such as targeted myocardium tissue and endocardium tissue, with demonstrable sparing of red blood cells, vascular smooth muscle tissue, endothelium tissue, nerves and other non-targeted proximate tissue.

Additionally, the electrode assembly on catheter 15 can be operated in a selected mode such as monopolar mode or bipolar mode. During monopolar operation of the catheter 15, an ablation electrode, a group of ablation electrodes, or the entire electrode assembly are configured as one of an anode or a cathode. None of the electrodes in the electrode assembly are configured as a the other of the cathode or the anode. Instead, the other of the cathode or the anode is provided in the form of a pad dispersive electrode located on the patient, typically on the back, buttocks, or other suitable anatomical location during electroporation. An electrical field is formed between an activated electrode of the electrode assembly and the pad dispersive electrode. During bipolar operation of the catheter 15, a first set of one or more electrodes of the electrode assembly, is configured as the anode and a second set of one or more electrodes of the electrode assembly, is configured as the cathode, to generate the electric field. In this example, a pad dispersive electrode is not used, and the electrical field is not extended in the patient's body, but rather through a localized portion of tissue proximate the electrode assembly. For example, the electrodes on the ablation electrode assembly are configured as the one of the anode or cathode and electrodes on the shaft proximate the distal end are configured as the other of the cathode or anode.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A medical device comprising:
a tubular outer shaft having a proximal end and an opposite distal end, and a lumen extending between the proximal end and the distal end;
an electrode assembly extending distally from the distal end of the outer shaft, the electrode assembly having a distally located central hub portion and a plurality of splines, each of the plurality of splines including a distal end portion extending from the central hub portion, and a proximal end portion attached to the outer shaft, at least one electrode located on each of the plurality of splines; and
an asymmetrical marker located on the outer shaft, the asymmetrical marker including a transverse member, a distal flag extending distally from the transverse member, and a proximal flag extending proximally from the transverse member;
wherein the distal flag is aligned with one of the plurality of splines and the proximal flag is aligned with a second of the plurality of splines.

2. The medical device of claim 1, wherein the asymmetrical marker is formed of a radiopaque material viewable under imaging.

3. The medical device of claim 1, wherein the transverse member is curved to conform to the outer shaft.

4. The medical device of claim 1, wherein the transverse member is a ring that circumscribes the outer shaft.

5. The medical device of claim 1, wherein the one of the plurality of splines is adjacent the second of the plurality of splines.

6. The medical device of claim 5, wherein the second of the plurality of splines is located clockwise from the one of the plurality of splines.

7. The medical device of claim 5, wherein the second of the plurality of splines is located counterclockwise from the one of the plurality of splines.

8. The medical device of claim 1, wherein at least one spline is between the one of the plurality of splines and the second of the plurality of splines.

9. The medical device of claim 1, wherein the outer shaft includes a sensor, and the distal flag is offset from the sensor.

10. The medical device of claim 1, wherein the electrode assembly includes an expanded configuration, and the distal flag terminates proximally of a bend of the one of the plurality of splines in the expanded configuration.

11. The medical device of claim 1, wherein the asymmetrical marker is at least partially embedded in the outer shaft.

12. The medical device of claim 1, wherein the distal flag or the proximal flag includes at least one protrusion.

13. The medical device of claim 1, wherein the distal flag is asymmetric along its length from the transverse member to a distal end.

14. The medical device of claim 1, wherein the proximal flag is asymmetric along its length from the transverse member to a proximal end.

15. The medical device of claim 1, wherein the proximal flag is symmetric along its length from the transverse member to a proximal end, and the distal flag is symmetric along its length from the transverse member to a distal end.

16. An electrosurgical medical device, the device comprising:
an outer shaft having a proximal end and an opposite distal end;
an electrode assembly extending distally from the distal end of the outer shaft, the electrode assembly having a distally located central hub portion and a plurality of splines, each of the plurality of splines including a distal end portion extending from the central hub portion, and a proximal end portion attached to the outer shaft, at least one electrode located on each of the plurality of splines; and
an asymmetrical marker at least partially embedded in the outer shaft, the asymmetrical marker including a transverse member, a distal flag extending distally from the transverse member, and a proximal flag extending proximally from the transverse member;
wherein the distal flag is aligned with one of the plurality of splines and the proximal flag is aligned with a second of the plurality of splines.

17. The medical device of claim 16, wherein the distal flag is asymmetric along its length from the transverse member to a distal end, or the proximal flag is asymmetric along its length from the transverse member to a proximal end.

18. The medical device of claim 16, wherein the proximal flag is symmetric along its length from the transverse member to a proximal end. and the distal flag is symmetric along its length from the transverse member to a distal end.

19. An electrosurgical medical device, the device comprising:
a tubular outer shaft having a proximal end and an opposite distal end, and a lumen extending between the proximal end and the distal end;
an electrode assembly extending distally from the distal end of the outer shaft, the electrode assembly having a plurality of splines, each of the plurality of splines including at least one electrode and a proximal end portion attached to the outer shaft; and
an asymmetrical marker located on the outer shaft, the asymmetrical marker including a transverse member including a curve configured to conform to the outer shaft, a distal flag extending distally from the transverse member, and a proximal flag extending proximally from the transverse member;
wherein the distal flag is aligned with one of the plurality of splines and the proximal flag is aligned with an adjacent spline.

20. The medical device of claim 19, wherein the asymmetrical marker is formed of a radiopaque material viewable under imaging.
